(19) United States
(12) Patent Application Publication
(10) Pub. No.: US 2010/0004637 A1
(43) Pub. Date: Jan. 7, 2010

(54) System and method for implantation of devices having unknown biocompatible materials

(75) Inventor: John H. Erickson, Plano, TX (US)

Correspondence Address: ST. JUDE MEDICAL NEUROMODULATION DIVISION, 6901 PRESTON ROAD, PLANO, TX, 75024, US

(73) Assignee: Advanced Neuromodulation Systems, Inc., Plano, TX (US)

(21) Appl. No.: 10/966,360

(22) Filed: Oct. 15, 2004

Related U.S. Application Data
(60) Provisional application No. 60/512,124, filed on Oct. 17, 2003.

(51) Int. Cl. A61M 5/168; A61K 9/22

(52) U.S. Cl. 604/891.1; 604/256

(57) ABSTRACT

There is disclosed a system and method for allowing use within a body of devices having material not known for its biocompatibility with the body. In one embodiment, a magnetically controlled solenoid/valve is used where portions of the valve are directly in contact with compositions that are to be delivered to a target site. Advantage is taken of an existing solenoid/valve having chromium alloy parts by coating the portions of the valve that contact the deliverable composition with a known biocompatible material having good wear resistance. In one embodiment, titanium nitride (TiN) is used as the coating material.

### RELATED APPLICATIONS

This application claims priority to Provisional Application Ser. No. 60/512,124 entitled “SYSTEM AND METHOD FOR IMPLANTATION OF DEVICES HAVING UNKNOWN BIOCOMPATIBLE MATERIALS, filed Oct. 17, 2003, the disclosure of which is hereby incorporated herein by reference.

### TECHNICAL FIELD

This invention relates to systems and methods for implantation of devices having unknown biocompatible materials and more particularly to human implantable fluid valves and more specifically to such valves in which a portion of the valve having unknown bio-compatibility is contained within the fluid flow.

### BACKGROUND OF THE INVENTION

In many situations medication, or other liquid material, is delivered internal to the human body directly to a particular target location. For example, it has become accepted practice to deliver pain medication directly to a spine using a pump implanted within the body. These pumps may operate on constant pressure preset prior to insertion into the body. Such constant pressure pumps are designed to deliver a premeasured amount of medication per unit of time.

In some situations, it is desirable to control the flow of medication by, for example, controlling the flow rate of the medication. This can be accomplished by changing the opening through which the liquid must pass, thereby changing the volume of medication sent to the target site. One design for metering such delivery is to use a solenoid which magnetically controls a valve located directly in the flow stream of the liquid.

Such devices suited for this task might use plungers of unknown bio-compatibility which may potentially allow traces of potentially harmful substances to be delivered to the target site, if the plunger were to be in contact with the medication. While, often, these substances have not proven to be medically significant they likewise have not been shown to be harmless. Tests to prove lack of harm are time consuming and expensive. Because of extensive testing, it has not been feasible to use “off-the-shelf” devices for implantation because of the unknown risk factors such devices might present.

### BRIEF SUMMARY OF THE INVENTION

There is disclosed a system and method for allowing a user to determine which components within a device are of biocompatible concern and to isolate such components. In one embodiment, a magnetically controlled solenoid/valve is used where the valve is directly in contact with compositions that are to be delivered to a target site. Advantage is taken of an existing solenoid/valve having a chromium plunger by coating the portions of the plunger that contact the deliverable composition with a known biocompatible metallic material having good wear resistance. In one embodiment, titanium nitride (TiN) is used as the coating material.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiment disclosed may be readily utilized as a basis for modifying or designing other structures for carrying out the same purposes of the present invention. It should also be realized that such equivalent constructions do not depart from the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 illustrates a valve shown in the open position;

FIG. 2 is a sectional view taken along section 2-2 of FIG. 1;

FIG. 3 illustrates a typical valve and pump system used for the delivery of medication to a human spine; and

FIG. 4 shows one embodiment of the distal end of the valve plunger.

### DETAILED DESCRIPTION OF THE INVENTION

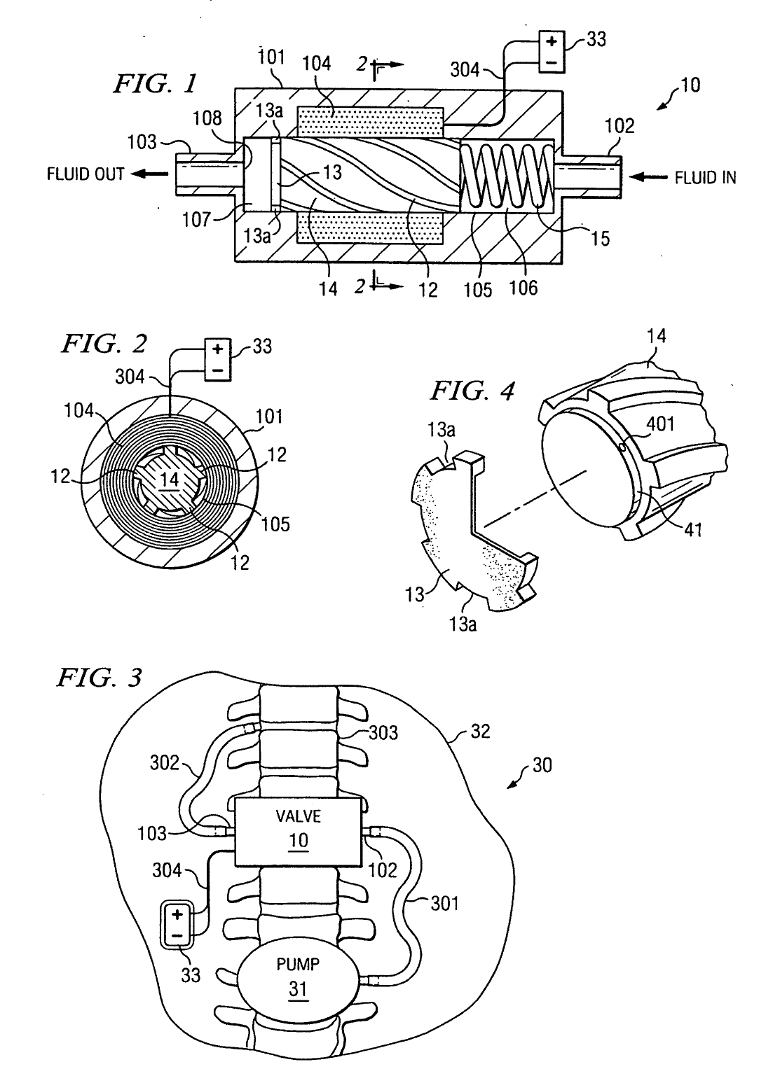

FIG. 1 shows valve 10 in the open position where voltage is applied to solenoid 104 forcing plunger 14 to the right (proximal end). Fluid arriving at input port 102 fills chamber 106 and passes along plunger 14 via channels 12. The fluid then passes around (or through) the ends of seal 13 (for example, via openings 13a) and enters chamber 107. The fluid then passes out of the valve via opening 108 and output port 103.

Plunger 14 is longitudinally positioned within a hollow core of housing 101 through the center of solenoid 104 essentially forming a first chamber 106 at the proximal end of the plunger and a second chamber 107 at the distal end. Spring 15 is positioned within chamber 106 and exerts longitudinal force on the proximal end of plunger 14 when the valve is open (plunger 14 moved to the right toward the proximal end). The force of spring 15 acting on plunger 14 is toward the distal end, when spring 15 is compressed.

When it is desired to stop or reduce the fluid flow, electrical voltage is removed from solenoid 104 allowing spring 15 to push plunger 14 to the left (toward the distal end) forcing the body of seal 13 into contact with opening 108 of output port 103 so as to prevent (or reduce) fluid from passing out of valve 10.

In operation, the voltage can be pulsed on and off so that the amount of fluid delivered at the output of port 103 is precisely controllable. The power source for valve 10 can be either internal to the body or external, and can be a battery (33, FIG. 3) or other voltage source with power and control delivered via wires (or wireless) 304. The pulsing of the solenoid can be hard wired controlled (via wires not shown) or can be wirelessly controlled. The valve can be designed to operate in the opposite manner such that when the voltage is applied the valve closes and when the voltage is removed the valve opens. However, for fail-safe purposes in most applications it would be desirable to have the valve close when power fails so that less and not more medication passes to the target site. Note that in some embodiments the seal could be designed to allow some fluid to flow even when in the “closed” position.

Spring 15 can be, for example, 316 stainless steel or any other material acceptable for implantable use directly into the liquid stream being delivered to the target site. Plunger 14, is material responsive to the magnetic fields created by solenoid 104. In the embodiment shown, plunger 14 is a chromium alloy, for example Cr¹⁸. The portion of plunger 14 and interior chamber walls that contact the material to be delivered to the target site is coated in a biocompatible material, such as titanium nitride (TiN). The coating material can be metallic or non-metallic as desired. Housing 101, which can be implantable in a human body, comprises material, such as 316 stainless steel which is known to be biocompatible for implant purposes where contact with the medication is not present. Seal 15 on the end of plunger 14 can be, for example, silicone, which is also a biocompatible material.

In one embodiment, the plunger is made of non-biocompatible or unknown biocompatible material, whose biocompatible properties are not well know. While analysis of each material's biocompatible property could occur, this involves expensive and time consuming testing, which may not provide ascertainable results. Such testing would delay introduction of materials into products and might not provide the surety needed to satisfy governmental approval for use.

Use of known biocompatible materials as a coating on parts having unknown biocompatiblility solves this problem. For one embodiment, titanium nitride (TiN) is used for the above mentioned properties, thereby allowing the valve to be produced at a relatively low cost without costly testing.

In operation, a user desiring to implant a device in a human (or in an animal) could select an off-the-shelf device having unknown biocompatible materials and then determine which portions of the selected device would cause problems if the material of the determined portions was not biocompatible with the human (or animal) body. Once these “potential” trouble portions are identified, the user would select a material that is known to be biocompatible and also known to be compatible with the desired function within the device. The potential trouble spots are then coated by plating or otherwise with the selected material. In some situations, different portions of the device could have different requirements depending upon the function to be performed and the implanted location of the device. Thus, as discussed herein, those portions of the device that are implanted under the skin may have a different biocompatibility requirement than does those portions of the device coming into contact with medication to be delivered or coming into contact with the blood supply of the patient.

In some embodiments, the liquid could flow on the outside of plunger 14 provided plunger 14 was supported within the hollow core of housing 101 and provided any surface that the liquid contacted was of a biocompatible material. This support, for example, could be a series of O-rings, each having holes therethrough so that the liquid could pass around the plunger while the plunger is supported within the O-rings. The liquid would then flow into chamber 107 and out of outlet 103 when the plunger is in the open position. When the plunger moves to the closed position, seal 13 would be forced against opening 108 thereby stopping (or reducing) the flow of liquid.

FIG. 2 is a sectional view taken along section 2-2 of FIG. 1. Housing 101 is shown around solenoid 104 which in turn surrounds plunger 14 and seal 13. Fluid channels 12 are shown around the outside of plunger 14. Note that the fluid channels can be spiraled, as shown in FIG. 1, or can be longitudinal along the plunger. These channels can be on the outside surface of plunger 14 or internal thereto, or a combination thereof.

FIG. 3 shows one illustrative system 30 in which catheter 302 delivers a measured amount of medication to human spine 303 within human body 32. Catheter 302 is connected to output port 103 of valve 10. Pump 31 is any pressure source which can deliver a measured amount of medication, under constant pressure if desired, to catheter 301 which, in turn, delivers the medication to input port 102 of valve 10. Valve 10, then is operational to meter the medication to the target site, or sites, within spine 303. In this embodiment, pump 31, valve 10 and catheters 301 and 302 are all implanted within human body 32. Pump 31 could also be, for example, a spring driven infusion pump of the type described in U.S. Pat. No. 4,772,263. Also, instead of a pump, or in addition thereto, a positive pressure reservoir can be used.

Since plunger 14 is constructed of a relatively hard material, such as chromium or a chromium alloy, the coating placed thereon should also be hard. Accordingly, the use of titanium nitride (TiN) which is a relatively brittle and hard material will work well. The thickness of the coated TiN, in one embodiment is between 3 microns and 5 microns but may vary depending on the application. The metallic coating could be deposited using well-known vapor depositing techniques or any other process. One method for depositing the coating would be to put a pin (or other holding device) through hole 401 (FIG. 4) at the distal end of plunger 14 for the purpose of holding the plunger during the vapor depositing process. At the end of the coating process, the plunger is removed from the depositing bath and the pin (or other holding device) is removed. The plunger will then be coated, except possibly where the pin (or other holding device) had been positioned.

As shown in FIG. 4, in one embodiment seal 13 then can be force fitted, or otherwise attached using adhesive or fasteners as desired, to circumferentially reduced portion 401 at the distal end of plunger 14 allowing the seal to effectively cover any portion of plunger 14 that was not fully coated. Platinum would be another know biocompatible material, however platinum does not have the durability as does titanium nitride.

By using titanium nitride, a known biocompatible material, and recognizing that it has the hardness required to coat a magnetic material, such as chromium, without interfering in the magnetic solenoid operation, a metering valve can be constructed from an essentially “off-the-shelf” valve that is human implantable, even though the “off-the-shelf” valve could not be so used without proper coating of the plunger.

While an in-line valve (liquid stopper) has been shown in one embodiment, any type of valve could benefit form the concepts taught herein. For example, a rotary valve could be used which rotates open and closed with the areas touching the liquid being coated with a biocompatible material. Also, while a human body has been shown, the concepts taught herein can be used for implantation in any body, including animals.

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

1.-6. (canceled)

7. A valve for use in controlling liquid flow situations where biocompatibility with said liquid is required, said valve comprising:
a passage through which said liquid can flow under pressure;
a liquid stopper positioned within said passage, said stopper blocking said passage to create proximal and distal portions; said stopper allowing liquid to flow in contact therewith from said proximal portion to said distal portion when said stopper is in an open position; and
a biocompatible metal coating on any surface of said stopper contacting said liquid, said surface having unknown biocompatibility without said coating.

8. The valve of claim 7 wherein said liquid flows in channels running along said stopper such that said liquid only contacts said stopper as said liquid passes between said portions.

9. The valve of claim 8 further comprising:
a solenoid for moving said stopper within said passage such that when said stopper is in a closed position sad fluid is restricted from passing out of said distal portion.

10. The valve of claim 7 wherein said coating is titanium nitride (TiN).

11. The valve of claim 10 wherein said stopper is a chromium alloy.

12. The valve of claim 10 wherein said solenoid is circumferentially around said stopper along the length of said stopper, said solenoid operable for moving said stopper in at least one direction.

13. The valve of claim 12 further comprising:
a spring within said proximal portion for forcing said stopper into a closed position, said spring in contact with liquid flowing within said chamber.

14. The valve of claim 13 wherein said spring is biocompatible.

15. The valve of claim 14 further comprising:
an input port for receiving liquid under pressure and for allowing said liquid to low into said proximal portion in contact with said spring; and
an output port for allowing liquid which has passed beyond said stopper to flow out of said distal portion, said output port having a portion for contact with said stopper such that when said stopper is displaced toward said output port said output port is at least sealed to partially restrict liquid form flowing out of said port.

16. The valve of claim 15 further comprising:
a seal attached to said stopper, said seal allowing liquid to flow into said distal portion when said stopper is in the open position and said seal operable for restricting flow of liquid out of said distal portion when said stopper is in the closed position.

17. The valve of claim 16 wherein said seal is biocompatible.

18. The valve of claim 7 wherein said stopper is a chromium alloy having plated thereon titanium nitride.

19. An implantable valve comprising:
a solenoid having a longitudinal direction and defining a hollow area;
a plunger within said hollow area, said plunger selectively displaceable along said longitudinal direction under control of a magnetic field created by said solenoid:
liquid passages defined within said hollow area are such that liquid passes from an inlet to an outlet, said liquid passages coated with a biocompatible metallic material in all places where said liquid contacts said liquid passages, said coating keeping said liquid from contacting any biocompatible unknown material; and
a seal for restricting said liquid flow when said plunger is longitudinally displaced toward said distal end.

20. The valve of claim 19 wherein said valve is an in-line valve.

21. The valve of claim 19 wherein said biocompatibly unknown material comprises, at least in part, said plunger.

22. The valve of claim 21 wherein said plunger is a chromium alloy.

23. The implantable valve in claim 19 wherein said seal is positioned within said hollow area at said outlet end of said plunger.

24. The implantable valve in claim 23 further comprising:
a spring positioned within said hollow area in the path of said fluid flow, said spring operable for exerting lateral force on said inlet end of said plunger, said lateral force toward said outlet end of said plunger.

25. The implantable valve in claim 19 wherein said metallic coating comprises titanium nitride (TiN).

26. The implantable in-line valve in claim 19 wherein said solenoid is contained within a housing, said housing comprising:
an inlet adapted for attachment to a first catheter for allowing liquid flowing within said catheter to enter said hollow area at said inlet end of said plunger; and
an outlet adapted for attachment to a second catheter for allowing fluid to flow out of said hollow area and into said second catheter under control of said seal.

27. The implantable valve in claim 26 wherein said first catheter is connected to a pressure source and said second catheter feeds a target site.

28-31. (canceled)

32. An in-line valve for implanting in a human for the delivery of medication, said valve comprising:
means within the medication flow for controllably interrupting said delivery, said interrupting means including:
means for preventing said medication from contacting any surface that is not known to be biocompatible; said preventing means comprising, at least in part, plated material.

33. The valve in claim 32 wherein said plated material is titanium nitride (TiN).

34. The valve in claim 33 wherein said interrupting means comprises:
a solenoid operable for moving a plunger along said medication flow.

35. The valve in claim 34 wherein said plunger is made of a chromium alloy.

36. The valve in claim 34 wherein said plunger has channels there along, said channels plated with said TiN.

37. A system of delivering medication to target sites within a human body, said system comprising:
a pressure source for moving measured amounts of said medication to said target site through catheters implanted in a human body;
a valve for controlling said measured amounts of medication, said valve comprising:
a plunger laterally displaced within said passage, said plunger blocking said passage to create first and second chambers; said plunger allowing liquid to flow in contact therewith from said first chamber to said second chamber, said liquid flowing in contact with said plunger; and
a device for moving said plunger laterally within said passage such that when said plunger is in a closed position said fluid is blocked from passing out of said distal chamber.

38. The system of claim 37 wherein said liquid flows in channels running longitudinally along said plunger such that said liquid only contacts said plunger as it passes between said chambers.

39. The system of claim 37 further comprising:
a biocompatible metal coating on any surface of said plunger contacting said liquid.

40. The system of claim 39 wherein said coating is titanium nitride (TiN).

41. The system of claim 40 wherein said plunger is a chromium alloy.

42. The system of claim 40 wherein said device comprises:
a solenoid circumferentially around said plunger along the length of said plunger, said solenoid operable for laterally moving said plunger.

43. The system of claim 42 further comprising:
a spring within said chamber at said proximal end of said plunger for forcing said plunger laterally within said chamber, said spring in contact with liquid within said chamber.

44. The system of claim 43 wherein said spring is biocompatible.

45. The system of claim 43 wherein said spring is coated with a biocompatible material.

46. The system of claim 43 wherein said chamber comprises:
an input port for receiving liquid under pressure and for allowing said liquid to flow into said first chamber in contact with said spring; and
an output port for allowing liquid which has passed along said plunger through said channel to flow out of said distal chamber, said output port having a portion for contact with said plunger such that when said plunger is laterally displaced toward said output port said output port is sealed to prevent liquid from flowing out of said port.

47. The system of claim 46 further comprising:
a seal at an end of said plunger, said seal allowing liquid to flow into said second chamber when said plunger is in the open position and said seal operable for preventing flow of liquid out of said second chamber when said plunger is in the closed position.
